(19) United States
(12) Plant Patent Application Publication
(10) Pub. No.: US 2013/0019363 P1
(43) Pub. Date: Jan. 17, 2013

(54) Hosta plant named 'Ben Vernooij'

(50) ;

(71) Applicant: Marco Fransen, Tel Aar (NL)

(72) Inventor: Marco Fransen, Tel Aar (NL)

(21) Appl. No.: 13/135,847

(22) Filed: Jul. 14, 2011

(51) Int. Cl. A01H 5/00

(52) U.S. Cl. PLT/353

(57) ABSTRACT

A new and distinct Hosta cultivar named ‘Ben Vernooij’ is disclosed, characterized by distinctive round foliage on thick petioles, vigorous plant habit and strong foliage, with good resistance to environmental stressors and large flowers. The near variety is a Hosta, typically used for outdoor ornamental purposes.

### LATIN NAME OF THE GENUS AND SPECIES

Hosta hybrid

### VARIETY DENOMINATION

‘BEN VERNOOIJ’

### BACKGROUND OF THE INVENTION

The new cultivar was discovered as an induced mutation using the known chemical colchicine, of Hosta hybrid ‘First Frost’ unpatented. ‘Ben Vernooij’ was discovered by the inventor, Marco Fransen, a citizen of the Netherlands in October of 2008, at a research facility in Ter Aar, The Netherlands. The intent of the breeding program was to create new tetraploid Hosta hyrids with strong foliage and vigorous plants.

Directly after discovery of the tetraploid mutation, the inventor initiated cultivation in tissue culture, during October of 2008, in a commercial laboratory in Iribov Heerhugowaard, the Netherlands. Subsequently ‘Ben Vernooij’ has been reproduced by micro-propagation and has shown that the unique features of this cultivar are stable and reproduced true to type through several generations.

### SUMMARY OF THE INVENTION

The cultivar ‘Ben Vernooij’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘Ben Vernooij’ These characteristics in combination distinguish ‘Ben Vernooij’ as a new and distinct Hosta cultivar:
1. Distinctive, round foliage on thick petioles
2. Thick foliage resistant to environmental stressors.
3. Vigorous plant.
4. Large flowers.

### COMPARISON TO PARENT

Plants of the new cultivar ‘Ben Vernooij’ are similar to plants of the parent; Hosta hybrid ‘First Frost’ in most horticultural characteristics, however, plants of the new cultivar ‘Ben Vernooij’ are more vigorous and produce thicker, stronger leaves and thicker petioles than the parent variety. Additionally, the new variety produces larger foliage and flowers than ‘First Frost’. Leaves of ‘Ben Vernooij’ have a wider yellow margin than ‘First Frost’.

### COMMERCIAL COMPARISON

‘Ben Vernooij’ can be compared to the commercial variety Hosta hybrid ‘Sleeping Beauty’, unpatented. The varieties are similar in most horticultural characteristics, however, plants of the new cultivar ‘Ben Vernooij’ are more vigorous and produce thicker, stronger leaves than ‘Sleeping Beauty. Additionally, the new variety produces larger foliage than ‘Sleeping Beauty’ and have foliage with a wider yellow margin than ‘Sleeping Beauty’.

### BRIEF DESCRIPTION OF THE PHOTOGRAPHS

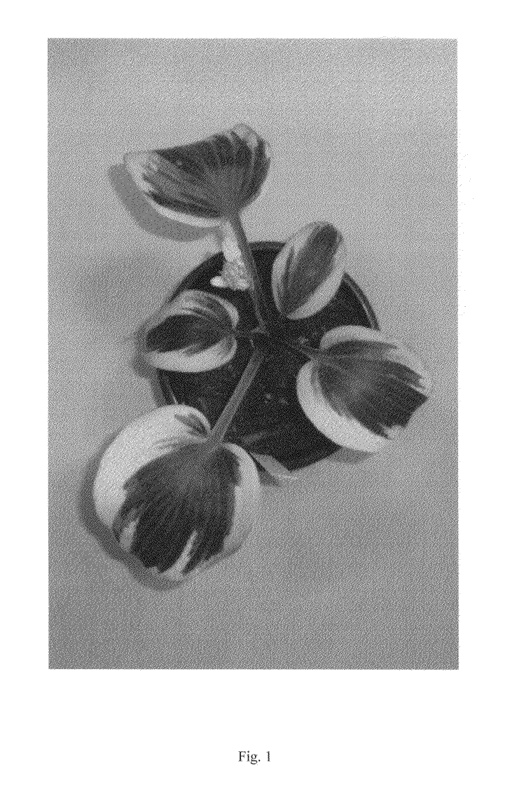

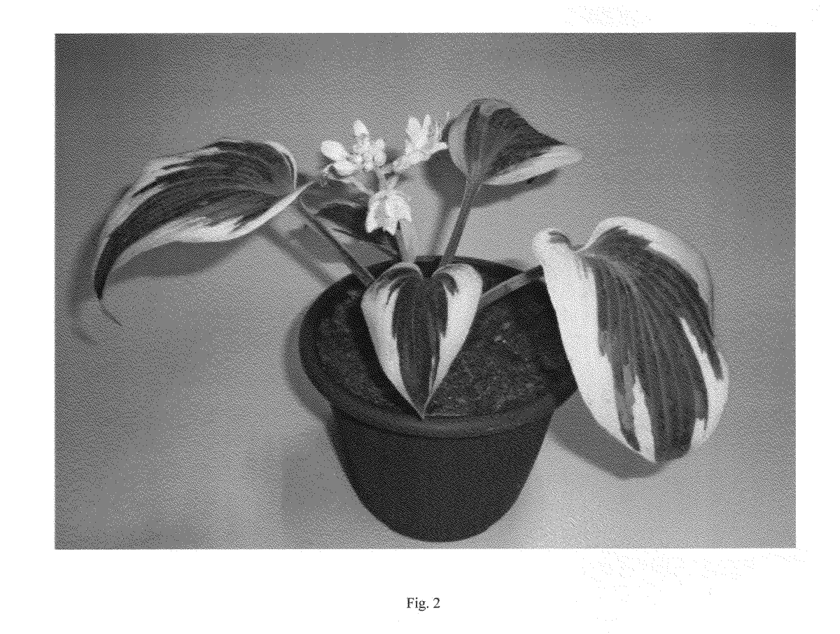

The accompanying photograph in FIG. 1 illustrates in full color a typical plant of ‘Ben Vernooij’ grown outdoors. This plant is approximately 6 months old, shown in a 1 liter pot. FIG. 2 illustrates a flowering plant of ‘Ben Vernooij’. The photographs were taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Colour Chart, 2001, except where general terms of ordinary dictionary significance are used. The following observations and measurements describe ‘Ben Vernooij’ plants grown in a commercial nursery in the Netherlands, from February of 2011 through June of 2011. Temperatures were 5° C. to 20° C. at night and 10° C. to 30° C. during the day. Measurements and numerical values represent averages of typical plant types. No growth regulators or special treatments were used.
Botanical classification: Hosta hybrid ‘Ben Vernooij’
Propagation:
Time to initiate roots: Approximately 14 to 21 at 23° C.
Root description: Fleshy with moderate side roots.
Plant:
Growth habit: Outwardly arching herbaceous perennial plant.
Height: Approximately 30 cm in a 1 liter pot.
Plant spread: Approximately 40 cm.
Growth rate: Moderate
Branching characteristics: Branches emerge directly from the roots.
Age of plant described: Approximately 4 months from a stage 3 tissue culture.
Foliage:
Leaf:
   Quantity per plant.—Average 20.
   Leaf type.—Simple, basal emerging.
   Average leaf length.—Approximately 30 cm.
   Average leaf width.—Approximately 18 cm, at widest point.
   Apex.—Acute.
   Base.—Cordate.
   Overall leaf shape.—Obovate.
   Overall leaf aspect.—Arching, reflexed back.
   Margin.—Entire.
   Texture of top surface.—Smooth.
   Texture of bottom surface.—Smooth.
   Appearance of top surface.—Slightly leathery.
   Appearance of bottom surface.—Slightly leathery.
   Pubescence.—No pubescence on leaves.
   Color.—Young foliage upper side: Near RHS Green 137A, margin Yellow 3B Young foliage under side: Near RHS Green 137A, margin Yellow 3B. Mature foliage upper side: Near RHS Green 137A, margin Yellow 2B Mature foliage under side: Near RHS Green 137A, margin Yellow 2B.
   Venation.—Type: Pinnate. Venation coloration upper side: Indistinguishable from foliage color. Venation coloration under side: Indistinguishable from foliage color.
Leaf petiole:
   Length.—Approximately 20 cm.
   Width.—Approximately 2.3 cm at widest point.
   Color.—Upper side: Near RHS Green 137B Under side: Near RHS Green 137B.
   Strength.—Very strong.
Other foliage characteristics: Thick foliage, resistant to mechanical damage and environmental stressors.
Flower:
Natural flowering season: Late Spring into Summer
Inflorescence type and habit: Typically — Single, campanulate arranged in racemes
Rate of flower opening: Lowest flower opens first, all flowers have opened in approx. 2 weeks
Flower longevity on plant: Approx. 2 weeks
Quantity of flowers: Average: 11 flowers per raceme
Raceme size:
   Height.—Approximately 5.2 cm, from lowest flower to top of Inflorescence.
   Width.—Approximately 7.1 cm.
Flower:
   Perianth.—Arrangement: Campanulate.
   Overall flower size.—Length: Approximately 6.5 cm Width: Approximately 2.6 cm at widest point.
Color: Tepals:
   When opening.—Outer surface: Near RHS White N155A, tinged near RHS Violet 85C Inner surface: Near RHS Violet 85B, base near RHS N155A, tube a color in between RHS Violet 85C and 85D.
   Fully opened.—Outer surface: Near RHS N155A, tinged near RHS Violet 85D Inner surface: Near RHS 85B, base near RHS N155A, tube near RHS 85D Color Changes when Aging: Near RHS N155A.
Sepals or bracts:
   Quantity.—One bract is placed under each individual flower.
   Shape.—Ovate, strongly concave.
   Length.—Approximately 2.3 cm.
   Diameter.—Approximately 0.5 cm.
   Color.—Outer surface: Near RHS Green 143C, base near RHS Yellow-Green 144D Inner surface: Near RHS 143C Color Changes when Aging: Near RHS Greyed-Orange 164B.
Bud:
   Shape.—Obovate.
   Length.—Approximately 2.7 cm.
   Diameter.—Approximately 0.8 cm.
   Color.—Near RHS NN155C, slightly tinged Purple 76C.
Pedicels:
   Length.—Approximately 0.6 cm.
   Width.—Approximately 0.2 cm.
   Aspect.—Placed to the petiole in an average angle of 30° (0°=horizontal), curved downward in an average angle of 20° (0°=horizontal).
   Color.—Near RHS NN155D.
Scape:
   Length.—Approximately 17.5 cm.
   Width.—Approximately 0.35 cm.
   Angle.—Near vertical.
   Strength.—Strong.
   Color.—Near RHS Yellow-Green 144A, covered with a thin waxy layer, near RHS Greyed-Green 190C.
Fragrance: No
Reproductive organs:
Stamens:
   Number.—Average 6.
   Filament length.—Approximately 5.3 cm.
   Filament color.—Near RHS Yellow-Green 145D.
   Anther length.—Approximately 0.6 cm.
   Anther color.—Near RHS Greyed-Orange 176C.
   Anther shape.—Oblong, dorsifixed.
   Pollen.—Moderate, coloured near RHS Yellow-Orange 17A.
Pistil:
   Number.—1.
   Length.—Approximately 5.7 cm.
   Style color.—Near RHS Greyed-Green 157C, base near Yellow-Green 145D.
   Stigma.—Shape: Club-shaped Color: Near RHS White N155D.
   Ovary color.—Near RHS Yellow-Green 145A.
Other characteristics:
Disease resistance: Better resistance to diseases or pests has been observed in this variety, due to the strong, thick foliage.
Temperature tolerance: Hardy perennial, tolerates a temperature range from approximately −30° C. to at least 40° C.
Drought tolerance: No tolerance for drought.

What is claimed is:

1. A new and distinct cultivar of Hosta plant named ‘Ben Vernooij’ as herein illustrated and described.
